(19) United States
(12) Patent Application Publication
(10) Pub. No.: US 2023/0049289 A1
(43) Pub. Date: Feb. 16, 2023

(54) ORALLY ADMINISTRABLE INTRAGASTRIC BALLOON

(71) Applicant: KEYRON LTD, London (GB)

(72) Inventor: Giorgio CASTAGNETO GISSEY, Roma (IT)

(73) Assignee: KEYRON LTD, London (GB)

(21) Appl. No.: 17/904,763

(22) PCT Filed: Feb. 19, 2021
(86) PCT No.: PCT/IB2021/051418

§ 371 Date: Aug. 22, 2022

(30) Foreign Application Priority Data
Feb. 21, 2020 (IT) 102020000003665

(51) Int. Cl. A61F 5/00; A61L 31/18; A61L 31/10; A61L 31/00; A61L 31/04; A61L 31/14

(52) U.S. Cl. CPC A61F 5/0036; A61L 31/18; A61L 31/10; A61L 31/005; A61L 31/041; A61L 31/148

(57) ABSTRACT

The present invention relates to an orally administrable intragastric balloon encapsulated so that, once it is in the stomach, the capsules dissolves and increases its volume thanks to the presence, inside thereof, of a superabsorbent edible polymer able of absorbing the water contained in the stomach.

### TECHNICAL FIELD OF THE INVENTION

The present invention relates to an orally administrable intragastric balloon encapsulated so that, once it is in the stomach, the capsule dissolves and increases its volume thanks to the presence, inside thereof, of a superabsorbent edible polymer able to absorb the water contained in the stomach.

### STATE OF ART

The use of intragastric balloons as minimally invasive treatment for pathological obesity represents a valid alternative to the bariatric surgery.

Many studies have shown an excess body weight loss 6 months after the treatment between 32.1% and 55.6% or about 25% after 1 year. In most cases, this loss was maintained for a short period (6 months) or a medium-term period (1 year), whereas 23% of patients maintained the weight loss for a maximum of 5 years, if associated to diet, training and modification of the life style.

A recent revision of literature underlined the fact that many complications linked to the intragastric balloon are due to an improper indication. The gastric perforation was the most frequent complication, reported in 22 cases. Nine of these 22 cases (40.9%) occurred very early after implantation (2 hours up to 3 days) in patients with a clear counterindication for the balloon insertion, in particular for the gastric or bariatric surgery which was previously performed [9].

Another important point to avoid complications is the endoscopist experience. In fact, American Society for Metabolic and Bariatric Surgery refers that the structures accredited to perform the procedure operated only 1,003 of 5,744 balloons inserted in 2016 in the United States by experienced endoscopists.

The migration of a deflated balloon and the intestinal obstruction were often due to the postponement of the balloon removal by the patients after more than 6 months, to the lack of using inhibitors of the protonic pump (PPI) which acted prophylactically or to the filling-in of the valve dysfunction after positioning.

On the whole, the treatment with balloon remains a safe procedure with a minimum of complications. Several types of balloons, however, need to be inserted and removed under sedation by an endoscopist.

Up to now only two types of intragastric balloons are available, which can be inserted by swallowing a big gelatin capsule, commercially known as Obalon and Elipse. When the capsule containing the device Obalon reaches the stomach, the physician inflates the balloon with gas (a mixture of nitrogen-sulphur-hexafluoride) by connecting the hose to an inflation system. In order to be sure that the capsule is in the stomach, a radiography is performed before inflating the balloon. The balloon volume is 250 ml and it is possible to ingest up to 3 balloons at a time. The removal, after 6 months, is performed laparoscopically under sedation.

The balloon Elipse represents an evolution of Obalon since it is positioned in a similar way but it is inflated with 550 ml of fluid under X-ray or ultrasound control. After 4 months, an automatic release valve is opened and the fluid disperses and the thin wall of the balloon is ejected with faeces (Bazerbachi F et al. Clin Endosc. 2017 January; 50(1):42-50).

Both these devices then require complex procedures in order to perform their function. In the state of art then the need is much felt to provide a more effective intragastric balloon not having the problems of those described in the known art.

### SUMMARY OF THE INVENTION

The technical problem placed and solved by the present invention is then to provide a device for the weight loss allowing to obviate the drawbacks mentioned with reference to the known art. In particular it has to allow to lose weight in a safe, controllable way, without any surgical operation.

Such problem is solved by an intragastric balloon according to claim 1.

Preferred features of the present invention are set forth in the depending claims.

Hereinafter some advantages of the device according to the present invention with respect to the known art are reported:
In no phase of its use an endoscopist or a surgeon assistance is required;
A catheter outside the body is not required to inflate the balloon;
The reinflating is carried out with water ingested directly inside the stomach;
It can be used even as system for releasing a drug.

The present invention does not require a hose for the inflation from outside. Once it is in the stomach, the capsule dissolves and enlarges in a balloon thanks to the presence, inside thereof, of a superabsorbent edible polymer which increases its volume up to 1,000 times (or more) by absorbing the water contained in the stomach.

Since the balloon wall is edible and degrades gradually, in particular in any moment from 1 day and 12 months or more, according to the material, the balloon content, consisted of small pieces of edible hydrated polymer, is slowly ejected by proceeding through the intestine. The balloon wall degrades gradually or in a precise moment, decided upon the construction of the same in relation to the material composing it, in the gastric acid environment, by allowing the balloon to be ejected naturally through the gastrointestinal tract.

Other advantages, features and use mode of the present invention, will result evident from the following detailed description of some embodiments, shown by way of example and not for limitative purposes.

### BRIEF DESCRIPTION OF THE FIGURES

The figures of the enclosed drawings will be referred to, wherein:

FIG. 1 is a schematic representation of an embodiment of the invention

FIG. 2 is a schematic representation of an embodiment of the invention if in use

### DETAILED DESCRIPTION

The present invention relates to an orally administrable intragastric balloon comprising:
a semipermeable, biodegradable and edible membrane suitable for passing the gastric fluid and water, wherein at least one biodegradable and edible superabsorbent polymer is enclosed in said membrane;
an external encapsulation layer that surrounds said membrane, whereby when said intragastric balloon is administered orally said encapsulation layer is rapidly degraded in the stomach and the gastric fluid and water passing through said membrane increase the volume of said superabsorbent polymer.

In the present description under the expression “said encapsulation layer is rapidly degraded in the stomach” it is meant that the encapsulation layer, once the balloon is administered orally, the layer is degraded in a period of time comprised between 1 second and 4 hours, preferably between 1 minute and 2 hours.

The gastric balloon advantageously has at least one duct capable to avoid the gastric obstruction, for example due to a mechanical lock on or near pylorus, preferably at least two ducts. Such ducts are communicating therebetween and with outside of balloon. The ducts preferably will be arranged orthogonally therebetween and/or will extend for the whole balloon length. The diameter of the ducts could vary for example between 0.5 to 3 cm, preferably will be about 2 cm.

Advantageously the membrane like the walls of said ducts could be radiopaque by allowing the detection of the balloon on X-rays.

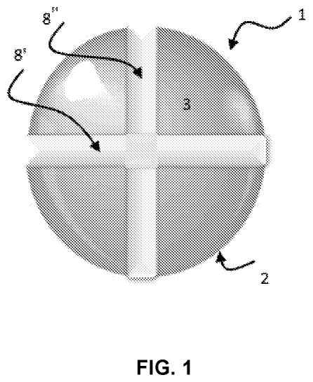

By making reference to the embodiment represented in FIG. 1, the balloon defined as a whole with number 1 has a substantially spherical shape, wherein the two ducts designated with numbers 8′ and 8″ are orthogonal to each other and extend for a length equal to the diameter of the substantially spherical balloon, by intersecting in the balloon centre.

In the present description under orally administrable intragastric balloons are meant devices which can be ingested and which expand to occupy a space inside an individual's stomach, by filling-in partially the stomach. The space occupied by the device physically limits the amount of food which the individual can eat, apart from creating a feeling of fullness which eliminates the individual's hunger. Consequently, the device can help an individual to lose weight. Additional applications are in the treatment of obesity, diabetes of type 2, Non-Alcoholic Fatty Liver Disease (NAFLD), Non-Alcoholic Steato-Hepatitis (NASH) and other correlated metabolic pathologies.

Thus, the herein described devices have volume and a shape capable, when ingested by a patient, of transiting down the esophagus from the mouth to the stomach.

For example, the balloon will be capsule-shaped or substantially spherical in shape.

The membrane (or herein also referred to as wall, denoted in figures with number 2) comprises of a biodegradable and edible polymeric film, i.e., a uniform layer of film that may be, for example, comprising a single component or a mixture of biopolymers (polysaccharides, proteins and lipids). For example, the algae-based edible films are non-toxic, degradable and biocompatible. Algae films have poor water vapor barrier proprieties due to the hydrophillic nature of algae. Thus, films consisting of algae (or marine macroalgae) allow water ingested and present in the stomach to pass inside the membrane.

According to an embodiment the membrane/wall will comprise kappa-carrageenan and/or alginate, these substances producing a film wherein kappa-carrageenan improves the moisture barrier and tensile properties of the alginate film. Further examples are algae/cellulose blend films obtained from solvent casting, they are elastic and permeable to water. For example, canola protein mixed with gelatin shows a tensile strength of 53.45 MPa.

By way of example, the materials or mixtures of the materials shown in Table A Otoni C G et al. could be used, Recent Advances on Edible Films Based on Fruits and Vegetables—A Review. Comprehensive Reviews in Food Science and Food Safety. 2017; 16: 1151-69.

The wall/membrane of the balloon could consist of other edible suitable material, namely a material with sufficient elasticity to resist expansion of the material, for example between 20 and 65 mPa, resistance to acids and peptidases, such that it is biodegradable in the stomach from 1 day to 24 months, preferably to be biodegradable in the stomach from 1 week to 12 months. The wall/membrane will for example have a tensile strength of at least 20 mPa, for example between 20 and 65 mPa, preferably about 50 MPa.

Between 0.1 and 10 grams (or more), preferably between 0.2 and 2 g of any superabsorbent polymer will be inserted within the membrane/wall. Once in the stomach, as the capsule dissolves, the water contained in the stomach will pass through the balloon membrane and increase the volume of the hydrophilic superabsorbent polymer by up to 200-5,000 times (or more). Superabsorbent polymers can absorb an amount of water approximately 100-100,000% (one hundred to one hundred thousand percent) (1-1,000 g/g) or more.

Examples of superabsorbent polymers (SAP) are hydrogels based upon biopolymers mainly deriving from vegetable extracts and/or animals and from cellulose. The latter, in fact, are called cellulose-based hydrogels. Natural hydrogels generally derive from polysaccharides and proteins. Cellulose and its derivative-based hydrogels are developed from polysaccharides. Citric acid is used to cross-link chitosan and other polysaccharides to create edible hydrogels. The natural-based superabsorbent polymers are those based on polysaccharides and polypeptides or proteins. Further SAP examples are alkylcellulose, methylcellulose, ethylcellulose, hydroxyethylcellulose, methylhydroxyethylcellulose, methylhydroxypropylcellulose, suitably cross-linked carboxymethylcellulose, for example with citric acid, borium and other cross-linking agents.

For example, the hydrogel networks including psyllium shells, which are natural polysaccharides and acrylic polymers, can be used in drug delivery devices. The powder volume increases in water and in acid solution to 1,400-5,000% or more in relation to the inflating medium. The acylated soy proteins can be another example. Advantageously, psyllium husk or acylated soy proteins can swell up to 5,000 times.

Preferably, the superabsorbent matrix will be a polyol polymer, in particular selected from cassava starch, maltitol, cellulose, glycerol, hydroped collagen, gelatin, dextrins or polyesters of sorbitol and dicarboxylic acids. In particular, dicarboxylic acids of various lengths of chain from C6 to C12. For example, C6 (adipic acid), C8 (suberic acid), C10 (sebacic acid), C12 (dodecanedioic acid) and longer chains. This polyester is biodegradable and can last in the stomach from 24 hours to 6 months, or longer, depending on the size of the superabsorbent polymer inserted into the membrane. The swelling of the superabsorbent polymer comprising the dicarboxylic acid can be more than 1,000 times its weight in the fluid.

The use as superabsorbent matrix of a polyol polymer esterified with carboxylic acids results to be particularly effective. Thus, a preferred embodiment is represented by an orally administrable intragastric balloon comprising a biodegradable and edible membrane suitable for passing gastric fluid and water, wherein at least one biodegradable and edible superabsorbent polymer is enclosed in said membrane and an encapsulation layer surrounds said membrane, whereby when said intragastric balloon is administered orally said encapsulation layer is rapidly degraded in the stomach and the gastric fluid and water passing through said membrane increase the volume of said superabsorbent polymer, wherein said superabsorbent polymer is a polyol selected from cassava starch, maltitol, cellulose, glycerol, hydroped collagen, gelatine, dextrins, sorbitol esterified with dicarboxylic acids. In particular, sorbitol esterified with dicarboxylic acids of various lengths of chain from C6 to C12. For example, C6 (adipic acid), C8 (suberic acid), C10 (sebacic acid), C12 (dodecanedioic acid).

The scheme for preparing a SAP for use in the balloons of the present invention is reported herebelow, wherein the hydroxylic groups of the cellulose are esterified with dicarboxylic acids C12, cellulose nanofibrils could be used too.

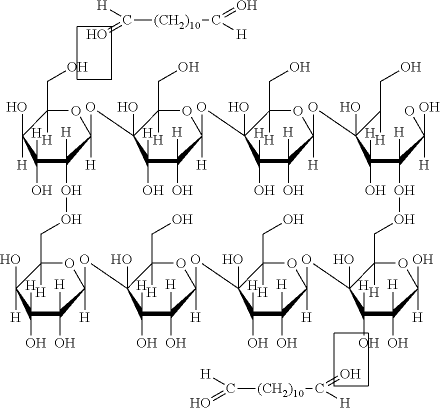

Cellulose is an exopolysaccharide existing in nature, consisting of one single type of monosaccharide, namely D-glucose linked with glycosidic bonds b-1, 4. It is mainly produced by plants; however, some animals (tunicates) and some microbes (bacteria and algae) also produce cellulose. Cellulose nanofibrils have a thickness of 5-10 nm and a length of several hundreds of nanometres. They are obtained by means of hydrolysis of different cellulosic sources with sulfuric acid or hydrochloric acid at controlled temperature.

In the above-schematized embodiment a dodecanedioic acid (C12) was used to create a polymer with cotton cellulose fibres. The cellulose esters and C12 were obtained in vitro in presence of sulphuric acid. The concentration of cellulose nanofibrils in the composite for example is 1, 2, 5, 10% w/w with respect to C12.

It is to be noted that herein the acid is a catalyst (regenerated at the end) and serves two purposes: first, it makes the carbonyl carbon a better electrophilic group and also allows the loss of H₂O as leaving group.

The membrane and its content of superabsorbent polymer are contained in a capsule which for example could be made of cellulose or any other suitable material dissolving in the stomach. Thus, for example capsules between 0.1-10 g, preferably between 0.5-2 g or more will be obtained.

According to a form of embodiment in the powder of the superabsorbent polymer one or more active principles for slimming could be mixed which will be slowly released in the stomach, for example Liraglutide, for example from 0.6 mg to a maximum of 1.8 mg released each day or hydrochloride monohydrate of sibutramine up to a maximum of 20 mg released per day, Orlistat up to a maximum of 120 mg released daily.

The intragastric balloons according to any one of the herein described embodiments will advantageously be joined by a thread, to form a plurality of smaller, and thus more easily degradable, capsules within the stomach. At the same time several single balloons could occupy a larger gastric volume.

The walls of the balloons are joined by a wire which connect them through the balloon membranes so as to be able to administer more than one capsule at the same time and to increase the occupied gastric volume.

For purposes of understanding the invention an administration mode of the balloon according to an administration form is reported hereinafter.

Insertion: One capsule is administered by oral route by means of insufflation with water (up to 500 ml or more) and the patient can assume up to 3 capsules (or more) in a row at maximum distance of 30-60 minutes each (or more).

It would be preferable to use ultrasound scanning or any other means, for example X-rays, to see the correct positioning of the device in the stomach.

Removal: once the balloon wall starts dissolving, the polymer will be removed gradually with feces for example in a period of 1 to 30 days, in particular between 1 and 15 days. In another embodiment, the digestion of the membrane is performed by ⅔ or more simultaneously by allowing the immediate ejection of the contained polymer with faeces.

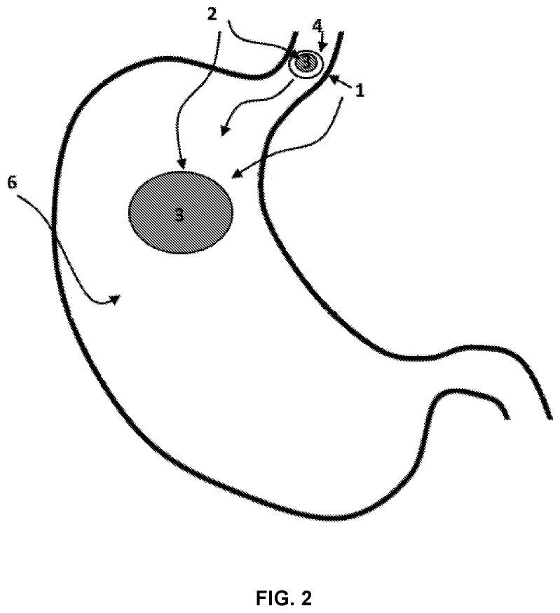

FIG. 2 is a schematic representation of an embodiment of the device if in use, the device is indicated as a whole with 1, whereas the membrane/wall and the absorbing core of the balloon are designated with 2 and 3 respectively.

### Examples

### Membrane/Balloon Wall Examples

Fruit: Papaya, Binding agent: gelatine or soy proteins and starch

relative humidity: 50±5%; tensile test, rigidity of the dynamometer 500.0 mm/min; tensile strength 20-30.5 MPa; elastic module 20-60 mPa; lengthening 13.1-28.1%.

Fruit: tomato Binding agent HM Pectin

relative humidity 33±2%; tensile test, rigidity of the dynamometer 7.5 mm/min; tensile strength 8.90-14.80 mPa; elastic module 187.20-65.20 mPa; lengthening 6.0-11.6%.

### SAP Polymer Examples

A superabsorbent polymer was obtained by using polyalcohols, such as sorbitol and chitin, and by making them to react with dicarboxylic acids having short chain with 4 to 6 carbon atoms. Although the reaction takes place spontaneously in acid environment, titanium butoxide increases the reaction yield. So obtained polyesters of polyols have a capability of absorbing water from 1000 to 1400 times their weight.

0.5 g of superabsorbent polymer were placed in the vegetable membrane folded several times to enter a 1.5-g capsule of gelatin. In vitro in acid environment (pH1-2) the gelatin capsule dissolves, water and acid penetrate through the membrane.

The balloon sizes increase in 1-2 hours reaching 500/700 ml of volume.

Under controlled conditions, temperature 37-40° C. and pH 1-2 and pepsin at concentration of 0.5-1 mg per millilitre to reproduce the stomach conditions the balloon started to have after 6 months and the membrane dissolved totally after 8 months.

The present invention has been described thus far with reference to some preferred embodiments. It is to be meant that other embodiments belonging to the same inventive core may exist, as defined by the protective scope of the here below reported claims.

1. An orally administrable intragastric balloon (1) comprising:
a biodegradable and edible membrane (2) suitable for passing the gastric fluid and water, wherein at least one biodegradable and edible superabsorbent polymer (3) is enclosed in said membrane;
an encapsulation layer (4) that surrounds said membrane, whereby when said intragastric balloon is orally administered, said encapsulation layer is rapidly degraded in the stomach and the gastric fluid and water passing through said membrane increase the volume of said superabsorbent polymer.

2. The intragastric balloon according to claim 1, further comprising at least one duct (8′,8″) to avoid gastric obstruction.

3. The intragastric balloon according to claim 2, wherein said balloon comprises two ducts (8′,8″), wherein said ducts are communicating with each other and with the outside of said balloon.

4. The intragastric balloon according to claim 2, wherein said two ducts (8′,8″), are orthogonal to each other and extend substantially over the entire length of said balloon.

5. The intragastric balloon according to claim 1, wherein:
said ducts have a diameter comprised between 0.5 and 3 cm; and/or
the walls of said ducts and/or said membrane are of a radiopaque material whereby said balloon is detectable on X-rays.

6. The intragastric balloon according to claim 1, wherein said membrane:
consists of a polymeric film biodegradable in the stomach in a period of time comprised between 1 day and 24 months; and/or
consists of a polymeric film with a tensile strength comprised between 20 and 465 mPa.

7. The intragastric balloon according to claim 1, wherein said encapsulation layer is made of cellulose and/or gelatin.

8. The intragastric balloon according to claim 1, wherein said absorbent polymer:
consists of a biodegradable polymer in the stomach in a period of time comprised between 1 day and 24 months; and/or
consists of a hydrophile biodegradable polymer able to increase its volume in presence of water from 10 to 1000 times.

9. The intragastric balloon according to claim 1, wherein said balloon before the oral administration has a weight comprised between 0.1 and 10 g.

10. The intragastric balloon according to claim 1, wherein said membrane consists of a material selected from kappa-carrageenan and/or alginate and/or Papaya and/or Papaya bound with gelatin and/or soy and/or starch proteins and/or tomato bound to HM pectin and/or mixture of algae/cellulose and/or cellulose and/or cellulose esters with dicarboxylic acids and/or cellulose esters with dicarboxylic acids with a chain comprised between C12 and C26.

11. The intragastric balloon according to claim 1, wherein said superabsorbent polymer consists of a polyol esterified with a dicarboxylic acid.

12. The intragastric balloon according to claim 1, wherein said superabsorbent polymer consists of cellulose esterified with dicarboxylic acids with a chain length comprised between C6 and C12.

13. The intragastric balloon according to claim 12, wherein the cellulose is in a concentration by weight with respect to the dicarboxylic acids comprised between 1 and 10% w/w.

14. A plurality of intragastric balloons according to claim 1, wherein said balloons are bound by a wire so as to form a plurality of capsules more easily degradable inside the stomach.

15. The intragastric balloon according to claim 5, wherein said ducts have a diameter of about 2 cm.

16. The intragastric balloon according to claim 6, wherein the polymeric film is biodegradable in the stomach in a period of time between 1 week and 12 months.

17. The intragastric balloon according to claim 9, wherein said balloon before the oral administration has a weight comprised between 0.5 and 2 g.

18. The intragastric balloon according to claim 11, wherein said superabsorbent polymer consists of a polyol esterified with a dicarboxylic acid with a chain length comprised between C6 and C12.

19. The intragastric balloon of claim 12, wherein said superabsorbent polymer consists of cellulose esterified with dicarboxylic acids with a chain length comprised between C6 and C12 and wherein said cellulose is in the form of nanofibrils.
